(19) United States
(12) Patent Application Publication
(10) Pub. No.: US 2023/0371807 A1
(43) Pub. Date: Nov. 23, 2023

(54) ILLUMINATION DEVICE FOR TEAR FILM LIPID LAYER ON OCULAR SURFACE ATTACHED TO CORNEAL TOPOGRAPHY

(71) Applicant: Yi WANG, ChongQing (CN)

(72) Inventor: Yi WANG, ChongQing (CN)

(21) Appl. No.: 17/748,359

(22) Filed: May 19, 2022

(51) Int. Cl. A61B 3/00; A61B 3/107; A61B 3/10

(52) U.S. Cl. CPC A61B 3/0008; A61B 3/107; A61B 3/101

(57) ABSTRACT

The utility model relates to an illumination device for tear film lipid layer on ocular surface. The illumination device for tear film lipid layer on ocular surface attached to corneal topography, located at the lower part of the subject's eye to illuminate the surface of the lower half of the subject's eye surface cornea for observing the tear film lipid layer; the illumination device comprises a rotating part and an illumination body, the rotating part is hinged on the corneal topography optical body, the illumination body is installed on the rotating part, and the illumination body light source is a surface light source; the light emitted by the light source of the eye surface tear film lipid layer illumination device is inclined relative to the eye axis direction, at an angle of ≥20°, which reduces the noise interference of the iris and other transparent parts behind the cornea, and improves the observation and imaging clarity.

### TECHNICAL FIELD

The patent relates to the field of special medical equipment, in particular to an eye surface tear film lipid layer lighting device attached to a corneal topographic map.

### BACKGROUND TECHNOLOGY

The eye is the most important organ in human senses. About 80% of the knowledge in the brain is obtained through the eye. Eyes should be used to read, read, picture, figure and enjoy the beautiful scenery.

Corneal topography can accurately analyse the changes of the morphology and curvature of the whole corneal surface of the eye. Specifically, the corneal surface of the eye is made into a local terrain and recorded and analysed by different methods. Its full name is the computer-aided corneal topography analysis system Computer assisted corneal topographic analysis system is a digital analysis of corneal morphology through computer image processing system, and the obtained information is represented by colour maps with different characteristics. It is called corneal topographic map because it looks like the ups and downs of topographic surface in geography. It can accurately measure and analyse the curvature of any point on the anterior surface of the whole cornea The measurement of corneal refractive power is a systematic and comprehensive quantitative analysis method to study the morphology of anterior corneal surface.

The corneal topography instrument consists of three parts: \textcircled{1} Placido's disc projection system. \textcircled{2} Real time image monitoring system. \textcircled{3} Computer image processing system.

Placido disc projection system projects 16˜34 concentric rings evenly onto the corneal surface from the centre to the periphery. The diameter of the central ring can be as small as 0.4 mm, and the ring can cover the whole cornea.

The corneal topography instrument can be used for the examination of dry eye diseases, such as the examination of tear film lipid layer on the ocular surface.

Tear film is a protective film on the surface of the human eye that separates the cornea from the air. The tear film consists of three layers, the inner layer is mucin, the middle layer is tear, and the outer layer is lipid layer. The distribution of lipid layer on the tear film surface is generally uneven, irregular and dynamic.

By blinking, people smear tears and lipid layer on the ocular surface secreting mucin to form a tear film. The tear film ruptures in about ten seconds under normal conditions. After a large number of ruptures, the tear film needs to be reconstructed by blinking; When the tear film changes, the rupture time will become faster. The human eye surface lacks tear film protection, and long-term direct contact with the air will produce discomfort, which is called dry eye.

Dry eye is the most common and ocular disease with high incidence rate. The incidence rate of 20˜36% in China is increasing. The development trend in recent years is increasing obviously with the increase of eye frequency. The study of this disease is of great importance to human health.

The causes of dry eye are divided into: mucin problem, tear problem and lipid layer problem. The problem of lipid layer causes dry eye, which is caused by insufficient or abnormal lipid layer, which cannot cover the tear surface well, make the tear film evaporate too fast and rupture too early, and cannot lock the water, resulting in dry eye.

The dry eye surface lipid layer was examined by corneal topography, and the circular light of placido disc projection system was used for illumination of the eye surface lipid layer. The disadvantages of using Placido disk projection system to examine the ocular surface lipid layer are as follows: a. the placido ring light irradiated by Placido disk projection system interferes with the observation of ocular surface lipid layer and affects the observation effect; b. Placido's disc projection system is about 110 mm away from the corneal surface of the ocular surface. It adopts scattered light surface illumination. Therefore, the distance is too far and the brightness is low, which makes the light shining on the ocular surface insufficient and the clear observation of the ocular surface lipid layer (the image is dim). The results can only be obtained by the doctor's analysis according to the captured image, rather than quantitative analysis.

### Patent Content

Aiming at the shortcomings of the prior art, the patent proposes an eye surface tear film lipid layer lighting device attached to the corneal topographic map. The specific technical scheme is as follows:

The utility model relates to an eye surface tear film lipid layer illumination device attached to the corneal topographic map, which comprises a corneal topographic map, which is characterized in that an eye surface tear film lipid layer illumination device is installed on the corneal topographic map, the eye surface tear film lipid layer illumination device is located at the lower part of the observer's eye, and illuminates the surface of the lower part of the cornea of the observer's eye surface, It is used to observe the lipid layer of tear film;

The eye surface tear film lipid layer illumination device comprises a rotating part and an illumination body, the rotating part is hinged on the corneal topography optical body, the illumination body is installed on the rotating part, and the illumination body light source is a surface light source.

Further, the light emitted by the light source of the eye surface tear film lipid layer illumination device is inclined relative to the eye axis direction, and the inclination angle is ≥20°.

Further, the rotating part drives the lighting body to rotate to the working position when in use and to the non-working position when not in use.

Further, the lighting body comprises a translucent flood plate, a lipid layer lighting device LED matrix lamp group and a lipid layer lighting device outer cover. The lipid layer lighting device LED matrix lamp group is installed on the inner surface of the lipid layer lighting device outer cover, and a translucent flood plate is installed on the lipid layer lighting device outer cover.

Further, the rotating part drives the lighting body to rotate to the working position when in use and to the non-working position when not in use.

Further, the lighting body comprises a translucent flood plate, a lipid layer lighting device LED matrix lamp group and a lipid layer lighting device outer cover, the lipid layer lighting device LED matrix lamp group is installed on the inner surface of the lipid layer lighting device outer cover, and a translucent flood plate is installed on the lipid layer lighting device outer cover.

Further, the illumination light emitted by the LED matrix lamp group of the lipid layer illumination device is diffused through the translucent flood board, transformed into a soft and uniform area light source, and mapped to the lower half of the cornea on the ocular surface of the eye.

The beneficial effects of this patent are:

First, the lighting device irradiates the corneal surface at a large tilt angle (tilt angle ≥20°), and there is no noise interference of Placido ring light irradiated by Placido disk projection system, which improves the observation and imaging clarity of ocular surface lipid layer.

Second, analyse the high-quality pictures obtained after irradiating the ocular surface lipid layer through the lighting device, which can accurately measure the thickness of different parts of the ocular surface lipid layer, and then calculate the average lipid layer thickness (LLT). Through these data, it is convenient to obtain a more accurate diagnosis conclusion.

Third, it is installed on the optical body of corneal topography and integrated with the optical body of corneal topography. The structure is compact. When in use, the lighting device turns over to the working position, and then turns over to the non-working position after use, which does not affect the realization of other functions of corneal topography.

### DESCRIPTION OF DRAWINGS

FIG. 1 is an exploded view of the lipid layer lighting device of the present patent;

FIG. 2 is a schematic diagram of the working position of the lipid layer lighting device of the present patent;

FIG. 3 is a general schematic diagram of the working position of the lipid layer lighting device of the present patent;

FIG. 4 is a schematic diagram of the non-working position of the lipid layer lighting device of the present patent;

FIG. 5 is a general schematic diagram of the non-working position of the lipid layer lighting device of the present patent;

The reference numerals in the figure are described as eyes 1 Eye axis direction (the subject's eyes are facing the front, and the direction of the eye axis) 2. The optical direction of the lipid layer lighting device 3. The rotation direction when the non-working position of the lipid layer lighting device turns to the working position 4-1. The rotation direction when the working position of the lipid layer lighting device turns to the non-working position 4-2. The translucent flood board 5, the outer cover of the lipid layer lighting device 6, the rotation mandrel of the lipid layer lighting device 7. Corneal topography Fig. optical body housing 8, Placido disk 9, Placido disk housing 10, magnet sheet 11, lipid layer lighting device wire cover 12, magnet 13, wire protective sleeve 14, lipid layer lighting device LED matrix lamp group 15 and lipid layer lighting device 16.

### SPECIFIC EMBODIMENTS

The preferred embodiment of the patent is described in detail below in combination with the accompanying drawings, so that the advantages and features of the patent can be more easily understood by those skilled in the art, so as to make a clearer and clear definition of the protection scope of the patent.

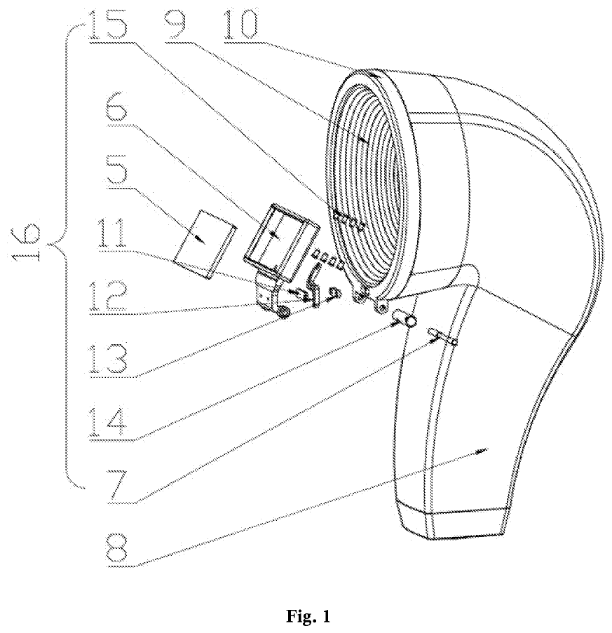

As shown in FIGS. 1 to 5, an eye surface tear film lipid layer illumination device is installed on the corneal topographic map, which is located at the lower part of the observer's eye 1; The light 3 emitted by the light source of the eye surface tear film lipid layer illumination device is inclined relative to the eye axis direction 2 (the front of the observed eye, the direction of the eye axis), and the inclination angle is 25°˜80°.

Two groups of LED matrix lamp groups 15 are arranged along the inner wall of the lipid layer lighting device housing 6, the translucent flood plate 5 is installed on the lipid layer lighting device housing 6, and the lower end of the lipid layer lighting device housing 6 is hinged with the bottom of Placido's disk housing 10.

The function of the translucent flood board 5 is to diffuse the light of the LED matrix lamp group 15 into a soft and uniform surface light source, so that the irradiation surface mapped to the lower half of the cornea on the ocular surface of the eye is uniform and clear.

The Placido disk housing 10 is hinged with the lipid layer lighting device housing 6 through the lipid layer lighting device rotating mandrel 7.

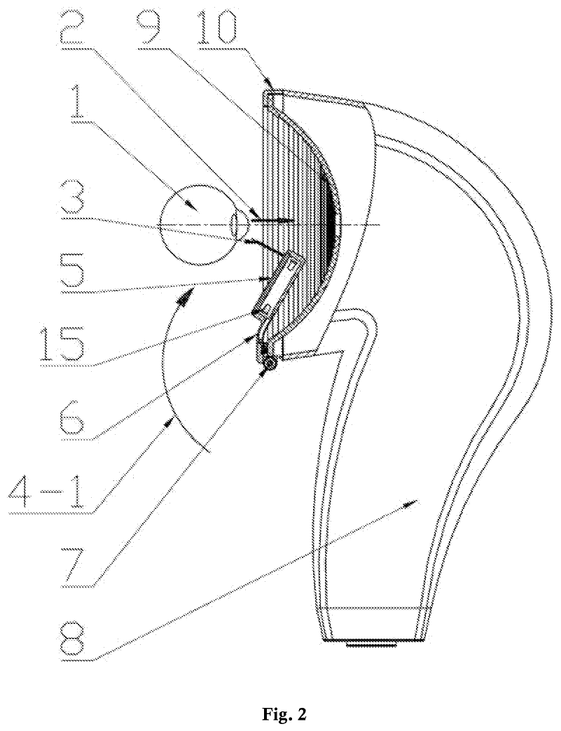

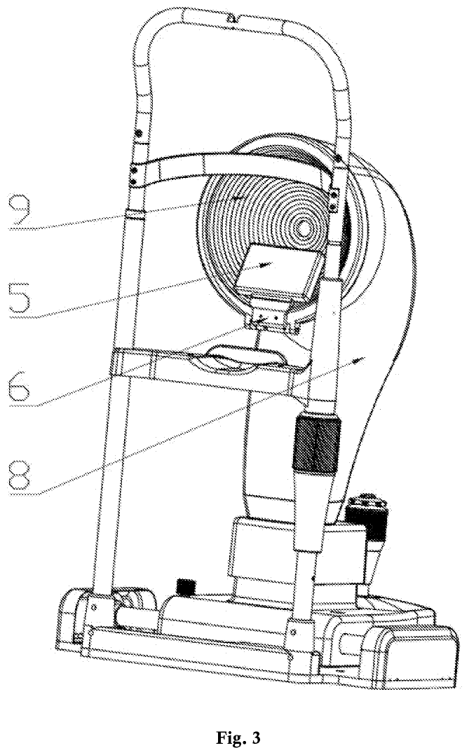

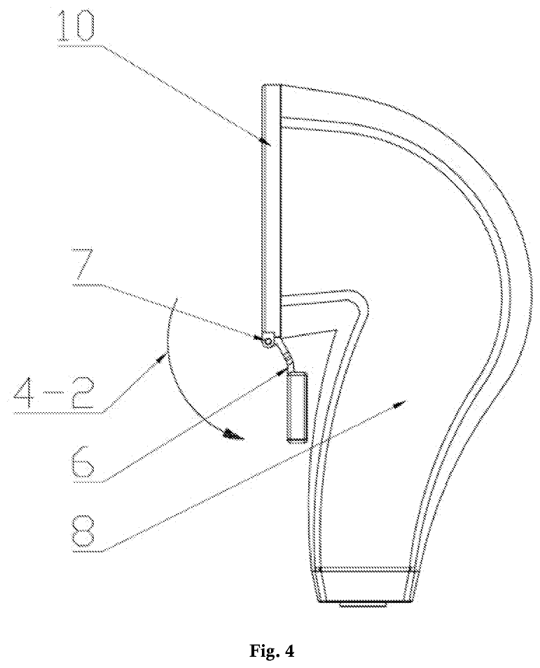

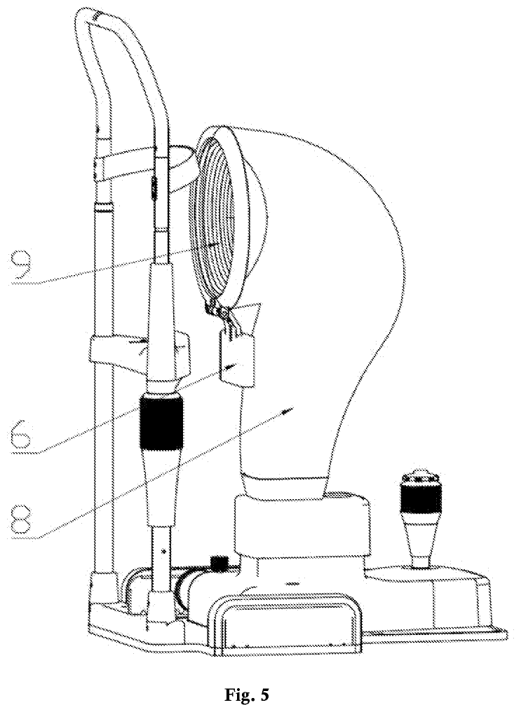

When the lipid layer lighting device 16 is used, the lipid layer lighting device 16 turns to the working position along the rotation direction 4-1 (FIG. 2, FIG. 3); after the lipid layer lighting device 16 works, the lipid layer lighting device 16 turns to the non-working position along the rotation direction 4-2 (FIG. 4, FIG. 5), which will not affect the realization of other functions of the corneal topography.

In the working position, the lipid layer lighting device 16 is fixed by the magnet 13 magnetically attracting the magnet sheet 11, wherein the magnet 13 is fixed on the placido disc 9 and the magnet sheet 11 is fixed on the lipid layer lighting device cover 6.

The illumination light emitted by the LED matrix lamp group 15 irradiates on the translucent flood board 5 to form an area light source. The area light source irradiates the corneal surface in the direction of the lower part of the eye surface of eye 1, then observes the lipid layer mapping image of the irradiated eye surface through the optical body, and calculates the lipid layer thickness of the irradiated part through different image colours.

In the preferred embodiment, an illumination device (16) for illuminating tear film lipid layer on an ocular surface attached to a corneal topography, characterized in that the illumination device is installed on the corneal topography, the illumination device comprises a rotating part (7) and an illuminator, the rotating part being hinged on the corneal topography optical body (8), and the illuminator is installed on the rotating part (7), and the illuminator comprises a light source which is a surface emitting light. The light emitted by the light source of the eye surface tear film lipid layer illumination device is inclined at an inclination angle relative to eye axis direction (2), said inclination angle is ≥20°. It is to be noted that the rotating part (7) drives the illuminator to rotate to a working position when in use and to a non-working position when not in use.

Further, the illuminator comprises a translucent flood plate (5), a lipid layer lighting device LED matrix lamp group (15) and a lipid layer lighting device cover (12), and the lipid layer lighting device LED matrix lamp group is installed on an inner surface of the lipid layer lighting device cover, the translucent flood plate (5) is installed on an outer surface of the lipid layer lighting device. The illumination light emitted by the LED matrix lamp group (15) of the lipid layer illumination device is diffused through the translucent flood plate (5), transformed into a soft and uniform area light source, and mapped to the lower half of the cornea on an eye surface.

For those skilled in the art, it is obvious that the patent is not limited to the details of the above exemplary embodiments, and the patent can be realized in other specific forms without departing from the spirit or basic features of the patent. Therefore, no matter from which point of view, the embodiments should be regarded as exemplary and non-restrictive. The scope of the patent is limited by the attached claims rather than the above description. Therefore, it is intended to include all changes within the meaning and scope of the equivalent elements of the claims in the patent. Any reference numerals in the claims shall not be regarded as limiting the claims involved.

In addition, it should be understood that although the description is described according to the embodiments, not each embodiment contains only one independent technical solution. This description of the description is only for clarity. Those skilled in the art should take the description as a whole, and the technical solutions in each embodiment can also be combined appropriately, Form other embodiments that can be understood by those skilled in the art.

We claim:

1. An illumination device (16) for illuminating tear film lipid layer on an ocular surface attached to a corneal topography, characterized in that the illumination device is installed on the corneal topography, the illumination device comprises a rotating part (7) and an illuminator, the rotating part being hinged on the corneal topography optical body (8), and the illuminator is installed on the rotating part (7), and the illuminator comprises a light source which is a surface emitting light.

2. The illumination device according to claim 1, wherein, the light emitted by the light source of the eye surface tear film lipid layer illumination device is inclined at an inclination angle relative to eye axis direction (2), said inclination angle is ≥20°.

3. The illumination device according to claim 1, wherein the rotating part (7) drives the illuminator to rotate to a working position when in use and to a non-working position when not in use.

4. The illumination device according to claim 1, wherein the illuminator further comprises a translucent flood plate (5), a lipid layer lighting device LED matrix lamp group (15) and a lipid layer lighting device cover (12), and the lipid layer lighting device LED matrix lamp group is installed on an inner surface of the lipid layer lighting device cover, the translucent flood plate (5) is installed on an outer surface of the lipid layer lighting device.

5. The illumination device according to claim 4, wherein the illumination light emitted by the LED matrix lamp group (15) of the lipid layer illumination device is diffused through the translucent flood plate (5), transformed into a soft and uniform area light source, and mapped to the lower half of the cornea on an eye surface.
